(19) United States
(12) Patent Application Publication
(10) Pub. No.: US 2024/0008728 A1
(43) Pub. Date: Jan. 11, 2024

(54) IMAGE PICKUP MODULE FOR ENDOSCOPE, AND ENDOSCOPE

(71) Applicant: OLYMPUS MEDICAL SYSTEMS CORP., Tokyo (JP)

(72) Inventor: Shunsuke HARI, Tokyo (JP)

(73) Assignee: OLYMPUS MEDICAL SYSTEMS CORP., Tokyo (JP)

(21) Appl. No.: 18/218,157

(22) Filed: Jul. 5, 2023

Related U.S. Application Data
(60) Provisional application No. 63/358,251, filed on Jul. 5, 2022.

(51) Int. Cl. A61B 1/05; A61B 1/06; A61B 1/00

(52) U.S. Cl. CPC A61B 1/053; A61B 1/0684; A61B 1/00137; A61B 1/00124; A61B 1/00103

(57) ABSTRACT

An image pickup module includes a first case including an image sensor and one or more of first connectors, and a second case including a power source and one or more of second connectors. A first one of the first connectors is electrically connected to a first one of the second connectors to form a power supply connection between the image sensor and the power source. A second one of the first connectors is electrically connected to a second one of the second connectors to form a ground connection between the image sensor and the power source.

### RELATED APPLICATION DATA

This application is based on and claims priority under 37 U.S.C. § 119 to U.S. Provisional Application No. 63/358,251 filed on Jul. 5, 2022, the entire contents of which are incorporated herein by reference.

### FIELD OF THE DISCLOSURE

The present disclosure relates to an image-pickup module for endoscope, an insertion portion for endoscope, and an endoscope that include single-use components and reusable components.

### BACKGROUND

Conventionally, endoscopes have been widely used, for example, in the medical field, the industrial field and the like. In general, an endoscope is configured by having an insertion portion in an elongated tube shape, and a distal end portion provided at a distal end of the insertion portion.

As an endoscope like this, there is known an endoscope of a so-called single-use type in a form of being discarded after being used only once. For example, Japanese Patent Application Laid-Open Publication No. 2013-502185 discloses a single-use type image pickup device. The single-use type image pickup device like this is discarded after being used once.

### SUMMARY OF THE DISCLOSURE

An image pickup module of one aspect of the present disclosure includes a first case including an image sensor and one or more of first connectors, and a second case including a power source and one or more of second connectors. A first one of the first connectors is electrically connected to a first one of the second connectors to form a power supply connection between the image sensor and the power source. A second one of the first connectors is electrically connected to a second one of the second connectors to form a ground connection between the image sensor and the power source.

### BRIEF DESCRIPTION OF THE DRAWINGS

FIG. 1 is a view showing an example of an entire configuration of an endoscope according to a first embodiment.

FIG. 2 is a view showing an example of a configuration of an image pickup module according to the first embodiment.

FIG. 3 is an enlarged view of a distal end configuring portion into which the image pickup module is fitted.

FIG. 4 is a view for explaining an example of sectional shapes orthogonal to a longitudinal axis of an insertion portion, of the image pickup module and the distal end configuring portion.

FIG. 5 is a view for explaining another example of the sectional shapes orthogonal to the longitudinal axis of the insertion portion, of the image pickup module and the distal end configuring portion.

FIG. 6 is a view for explaining an example of the sectional shapes orthogonal to a longitudinal axis of an insertion portion, of an image pickup module and a distal end configuring portion, in a front-viewing endoscope.

FIG. 7 is a view for explaining another example of the sectional shapes orthogonal to the longitudinal axis of the insertion portion, of the image pickup module and the distal end configuring portion, in the front-viewing endoscope.

FIG. 8 is a view showing an example of a configuration of an image pickup module according to a second embodiment.

### DETAILED DESCRIPTION

In general, single-use type endoscopes are assumed to be used only once, but have components that can be used a plurality of times depending on the components used. For example, an image pickup device is an expensive component, and if the image pickup device is discarded after being used only once, an environmental load and cost increase.

According to embodiments described below, it is possible to provide an image pickup module for endoscope, an insertion portion for endoscope, and an endoscope that can reduce an environmental load and cost by reducing the number of discarded components that are generated after being used once, in a single-use type endoscope.

Hereinafter, details of the embodiments will be described with reference to the drawings.

Note that the drawings based on the embodiments are schematic, relationships between thicknesses and widths of respective parts, ratios of the thicknesses, relative angles, and the like of the respective parts are different from actual relationships, ratios, relative angles, and the like. Even among the drawings, portions in which relationships and ratios of mutual dimensions are different are included.

### First Embodiment

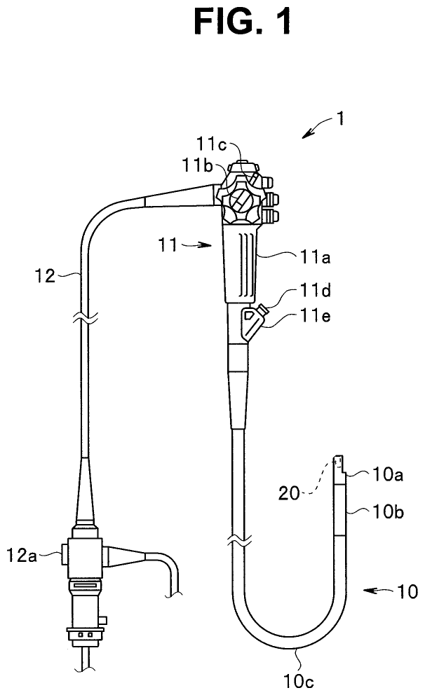

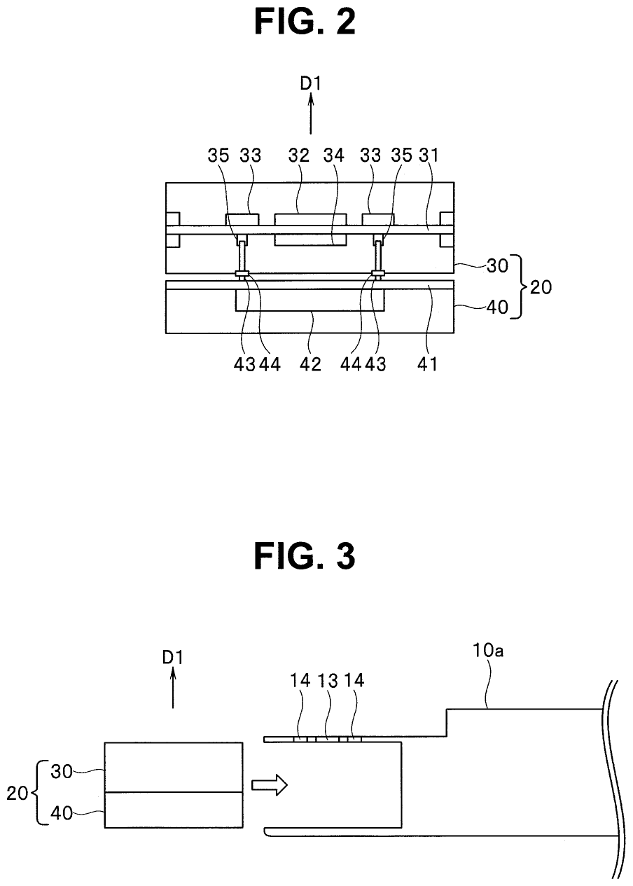

FIG. 1 is a view showing an example of an entire configuration of an endoscope according to a first embodiment. FIG. 2 is a view showing an example of a configuration of an image pickup module according to the first embodiment. FIG. 3 is an enlarged view of a distal end configuring portion into which the image pickup module is fitted.

As shown in FIG. 1, an endoscope 1 can be a single-use type endoscope, and includes an insertion portion 10, an operation portion 11, and a universal cable 12.

The endoscope 1 includes the insertion portion 10 including a distal end, and the image pickup module can be reversibly attached to the distal end of the insertion portion 10.

The insertion portion 10 is an elongated portion configured to be inserted into a body cavity of a subject. Note that the subject into which the insertion portion 10 is inserted is assumed to be a human body as an example, but the subject may be a living organism such as an animal, or may be a non-living thing such as a machine or a building, not limited to a human body.

The insertion portion 10 includes a distal end configuring portion 10a, an active bending portion 10b, and a flexible tube portion 10c, in order from a distal end side toward a proximal end side. In the distal end configuring portion 10a, an image pickup module (image pickup unit) 20 for picking up an image of a subject is arranged.

The image pickup unit 20 is disposed in the distal end configuring portion 10a of the insertion portion 10, and has an optical axis in a first direction D1 (see FIG. 2 and FIG. 3) that crosses a longitudinal axis of the insertion portion 10.

The active bending portion 10b is a bendable portion placed on a proximal end side of the distal end configuring portion 10a. The active bending portion 10b is configured to be bendable, for example, in two directions, or four directions, up, down, left, and right.

In the active bending portion 10b, a plurality of bending pieces are provided in a row to be swingable along the longitudinal axis of the insertion portion 10, and a bending wire is connected to a bending piece at a distal end. A proximal end of the bending wire is connected to a bending operation knob 11b of the operation portion 11.

When the active bending portion 10b is bent, a direction of the distal end configuring portion 10A changes, and an observation direction of the image pickup unit and an irradiation direction of illumination light from an LED 33 (see FIG. 2) change. The active bending portion 10b is also bent to improve insertability of the insertion portion 10 in a subject. The first case 30 can include the LED 33, and the LED is supplied with power from the power source 42 via the power supply connection.

The flexible tube portion 10c is a tube portion that is arranged on a proximal end side of the active bending portion 10b and has flexibility.

The operation portion 11 is arranged on a proximal end side of the insertion portion 10, and includes a grasping portion 11a, the bending operation knob 11b, a raising base (forceps elevator) operation lever 11c, and a treatment instrument insertion port 11d. The grasping portion 11a is a portion where an operator grasps the endoscope 1 with a palm. The bending operation knob 11b is an operation device for performing an operation of bending the active bending portion 10b by using a thumb or the like of a hand that grasps the grasping portion 11a. When the bending operation knob 11b is operated, the bending wire is pulled, and the active bending portion 10b is bent.

The raising base operation lever 11c is an operation device for performing an operation of raising a treatment instrument raising base disposed inside of the distal end configuring portion 10a by using a thumb or the like of the hand that grasps the grasping portion 11a. When the raising base operation lever 11c is operated, the raising wire is pulled, and the treatment instrument raising base is raised. Further, on the operation portion 11, various buttons for operating the endoscope 1 are also provided.

At a predetermined position of the grasping portion 11a, a protuberance portion 11e in a substantially triangular shape is formed. The treatment instrument insertion port 11d is provided in the protuberance portion 11e. The treatment instrument insertion port 11d is an opening that communicates with a proximal end of a treatment instrument channel provided inside of the insertion portion 10 and is for inserting a treatment instrument into the treatment instrument channel.

The universal cable 12 is extended from, for example, a side surface on a proximal end side of the operation portion 11, and is provided with a connector 12a for connecting to a light source apparatus and an endoscope control apparatus, at an extension end.

As shown in FIG. 2, the image pickup unit 20 is configured by a first case 30 and a second case 40.

The first case 30 has a substrate (first substrate) 31, an image pickup device (image sensor) 32, two LEDs 33, a transmitter 34, and two connection connectors (a plurality of first connectors) 35. The image pickup device 32 and the two LEDs 33 are disposed on a first surface of the substrate 31. The transmitter 34 and the two connection connectors 35 are disposed on a second surface opposite from the first surface of the substrate 31.

The first case 30 includes the image sensor 32 and the plurality of first connectors 35. The first case 30 can include the transmitter 34, and wherein the transmitter 34 is supplied with power from the power source 42 via the power supply connection. The transmitter 34 is configured to transmit an electrical signal outputted from the image sensor 32.

The second case 40 has a substrate (second substrate) 41, a power supply device (power source or battery) 42, two connection pins (a plurality of second connectors) 43, and two seal portions (a first seal and a second seal) 44. The two connection pins 43 are disposed on a first surface of the substrate 41. The power supply device 42 is disposed on a second surface opposite from the first surface of the substrate 41. The two seal portions 44 are provided respectively on outer peripheries of the two connection pins 43.

The second case 40 includes the power source 42 and the plurality of second connectors 43.

A first one of the first connectors 35 is electrically connected to a first one of the second connectors 43 to form a power supply connection between the image sensor 32 and the power source 42. A second one of the first connectors 35 is electrically connected to a second one of the second connectors 43 to form a ground connection between the image sensor 32 and the power source 42.

An exterior surface of the first case 30 includes a plurality of receiver channels. The first one of the first connectors 35 is located in a first channel of the plurality of receiver channels, and the second one of the first connectors 35 is located in a second channel of the plurality of receiver channels.

The first one of the second connectors 43 is a first connector pin inserted into the first channel and in contact with the first one of the first connectors 35, and the second one of the second connectors 43 is a second connector pin inserted into the second channel and in contact with the second one of the first connectors 35.

The first seal 44 can be connected to the first connector pin and the second seal 44 can be connected to the second connector pin.

The first seal 44 contacts a first surface portion of the first case 30 to seal the first channel, and the second seal 44 contacts a second surface portion of the first case 30 to seal the second channel.

The first case 30 and the second case 40 can extend along a longitudinal direction of the insertion portion 10, and the first case 30 and the second case 40 are stacked in a stacking direction that intersects the longitudinal direction. A longest dimension of the image sensor 32 and a longest dimension of the power source 42 both can extend along the longitudinal direction.

The first seal 44 can be connected to the first one of the second connectors 43, and the first seal 44 can contact a surface of the first case 30 to prevent a liquid from entering an interior of the first case 30. The first seal 44 can be connected to the first one of the first connectors 35, the first seal 44 can contact a surface of the second case 40 to prevent a liquid from entering an interior of the second case 40. The first seal 44 and the second seal 44 contact a surface of the second case 40 to prevent a liquid from entering an interior of the second case 40. At least one of the first seal and the second seal is an O-shaped ring.

The image pickup device 32 is, for example, an image sensor such as CCD or CMOS, and obtains an image of a subject. The LED 33 is an illumination device that irradiates the subject with illumination light. Note that on the substrate 31, the two LEDs 33 are provided, but one LED or three or more LEDs may be provided.

The transmitter 34 has an antenna and a modulation circuit. The transmitter 34 modulates data of an image of a subject obtained by the image pickup device 32 by the modulation circuit to generate a radio signal, and transmits the radio signal to an external receiver via the antenna. Note that the transmitter 34 is disposed on the second surface of the substrate 31, but may be disposed on the first surface of the substrate 31.

The external receiver is disposed in the endoscope control apparatus that is connected to the connector 12a. The receiver has an antenna, a demodulation circuit, and a processing circuit. The receiver receives the radio signal transmitted from the transmitter 34 via the antenna, and demodulates the received radio signal by the demodulation circuit. The processing circuit of the receiver outputs the data of the image of the subject that is demodulated by the demodulation circuit to a display apparatus.

Note that the external receiver is not limited to being disposed in the endoscope control apparatus that is connected to the connector 12a. For example, the external receiver may be disposed inside of the operation portion 11 of the endoscope 1. The receiver in the operation portion 11 receives data transmitted from the transmitter 34. The receiver transmits the received data to the endoscope control apparatus that is connected to the connector 12a by wire, via the universal cable 12.

The power supply device 42 is, for example, a primary battery such as an alkaline battery or a manganese battery, and supplies power to the first case 30 via the two connection pins 43. The power that is supplied to the first case 30 is supplied to the image pickup device 32, the LEDs 33, and the transmitter 34. In other words, the power supply device 42 configures a battery that supplies power to the image pickup device 32 of the first case 30.

The two connection pins 43 are pins for power supply and ground and are connected to the two connection connectors 35 of the first case 30. Thereby, the first case 30 and the second case 40 are connected to each other, and the image pickup device 32, the LEDs 33, and the transmitter 34 of the first case 30, and the power supply device 42 of the second case 40 are electrically connected. In other words, the connection pins 43 configure a joining portion that removably connects the image pickup device 32 of the first case 30 and the power supply device 42 of the second case 40. Note that assuming a failure of the connection connector 35 and/or the connection pin 43, the first case 30 and the second case 40 may have three or more connection connectors 35 and connection pins 43 respectively.

The two seal portions 44 are, for example, O-shaped rings or packings, and are disposed on outer peripheral portions of the respective connection pins 43. The seal portions 44 ensure watertightness of the first case 30 (seal the first case 30 in a watertight manner) when the first case 30 and the second case 40 are connected to each other.

The image pickup module 20 configured in this way is fitted into an inside of the distal end configuring portion 10a from a distal end side of the distal end configuring portion 10a as shown in FIG. 3. In the distal end configuring portion 10a, an observation window 13 and two illumination windows 14 are provided at positions that face the image pickup device 32 and the two LEDs 33 of the first case 30. The observation window 13 and the two illumination windows 14 are configured of cover glass, for example.

The image pickup module 20 is detached from the distal end configuring portion 10a after being used once, and is subjected to cleaning and disinfection. Subsequently, the first case 30 of the image pickup module 20 is reused, and the second case 40 is replaced after one use.

Note that cleaning and disinfection of the image pickup module 20 are performed in a state in which the second case 40 is connected to the first case 30, but if only the first case 30 is configured to keep watertightness alone, for example, cleaning and disinfection may be applied to only the first case 30 after the first case 30 is detached from the second case 40.

Since it is possible to reduce the number of discarded components generated after one use of the single-use type endoscope 1 by reusing the first case 30 like this, it is possible to reduce an environmental load. In addition, it is possible to reduce cost of the single-use type endoscope 1 by reusing the first case 30 having the image pickup device 32 that is an expensive component.

As a result, the single-use type endoscope 1 can reduce the environmental load and cost by reducing the number of the discarded components generated after being used once.

Note that the second case 40 has the power supply device 42 that is a primary battery, but may have, for example, a secondary battery such as a lithium ion battery. By using a secondary battery as the power supply device 42, it is possible to recharge and reuse the power supply device 42 after cleaning and disinfection, so that it is possible to further reduce the number of discarded components and it is possible to further reduce the environmental load and cost.

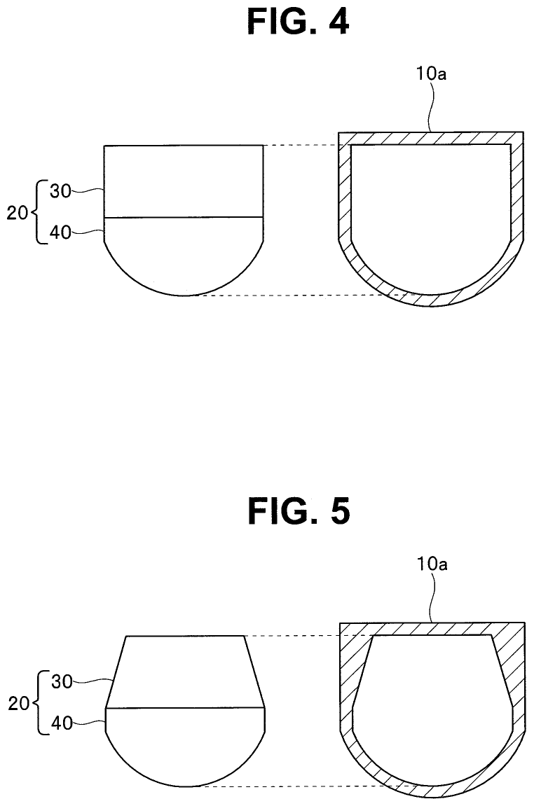

FIG. 4 is a view for explaining an example of sectional shapes orthogonal to a longitudinal axis of the insertion portion, of the image pickup module and the distal end configuring portion.

As shown in FIG. 4, the first case 30 of the image pickup module 20 has a rectangular sectional shape. The second case 40 of the image pickup module 20 has a sectional shape in a substantially semicircular shape.

The distal end configuring portion 10a has a sectional shape that allows the first case 30 and the second case 40 to be fitted into the distal end configuring portion 10a in a joined state.

Note that the sectional shapes of the image pickup module and the distal end configuring portion are not limited to the shapes in FIG. 4. FIG. 5 is a view for explaining another example of the sectional shapes orthogonal to the longitudinal axis of the insertion portion, of the image pickup module and the distal end configuring portion.

As shown in FIG. 5, the first case 30 has a trapezoidal sectional shape, and the second case 40 has a sectional shape in a substantially semicircular shape.

The distal end configuring portion 10a has a sectional shape that allows the first case 30 and the second case 40 to be fitted into the distal end configuring portion 10a in a joined state.

Note that the first case 30 has a bilaterally symmetrical trapezoidal shape, but may have a bilaterally asymmetrical trapezoidal shape. When the first case 30 has a bilaterally asymmetrical trapezoidal shape, an insertion direction at a time of the image pickup module 20 being fitted into the distal end configuring portion 10a is uniquely determined, so that it is possible to prevent incorrect assembly.

Further, the endoscope 1 of the present embodiment is a side-viewing endoscope, but is not limited to this, and may be a front-viewing endoscope.

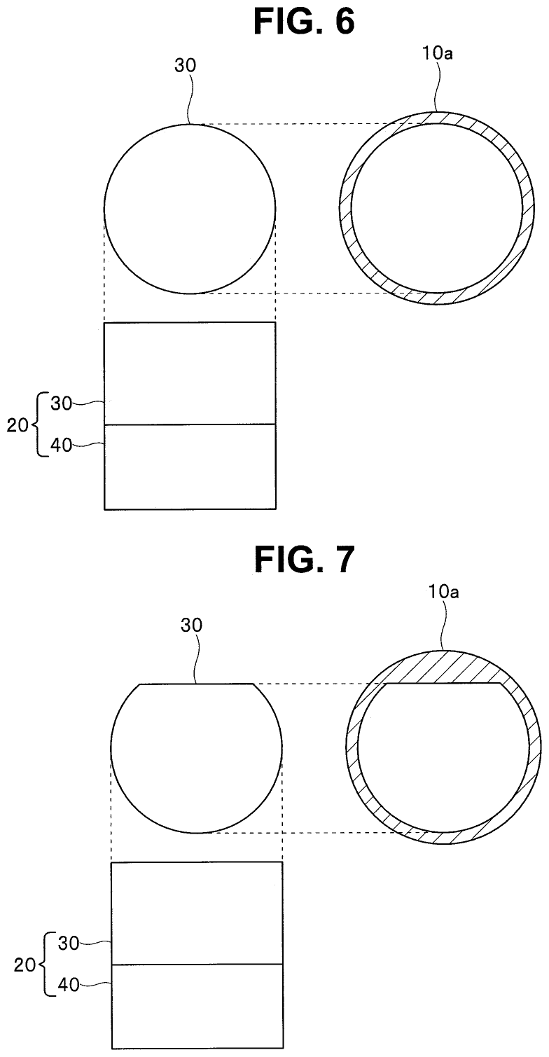

FIG. 6 is a view for explaining an example of sectional shapes orthogonal to a longitudinal axis of an insertion portion, of an image pickup module and a distal end configuring portion, in a front-viewing endoscope.

As shown in FIG. 6, a first case 30 has a circular sectional shape. A second case 40 also has a circular sectional shape. External shapes of the first case 30 and the second case 40 are circular column shapes.

A distal end configuring portion 10a has a sectional shape that allows the first case 30 and the second case 40 to be fitted into the distal end configuring portion 10a in a joined state.

An image pickup module 20 is fitted from the second case 40 into the distal end configuring portion 10a so that the first case 30 is disposed on a distal end side of the distal end configuring portion 10a.

FIG. 7 is a view for explaining another example of sectional shapes orthogonal to the longitudinal axis of the insertion portion, of the image pickup module and the distal end configuring portion, in the front-viewing endoscope.

As shown in FIG. 7, the first case 30 has a D-cut sectional shape. The second case 40 also has a D-cut sectional shape. External shapes of the first case 30 and the second case 40 are column shapes.

The distal end configuring portion 10a has a sectional shape that allows the first case 30 and the second case 40 to be fitted into the distal end configuring portion 10a in a joined state.

By a configuration like this, even when an image pickup device 32 is not disposed in a center of the first case 30, a position of the image pickup device 32 to the insertion portion 10 can be determined uniquely.

### Second Embodiment

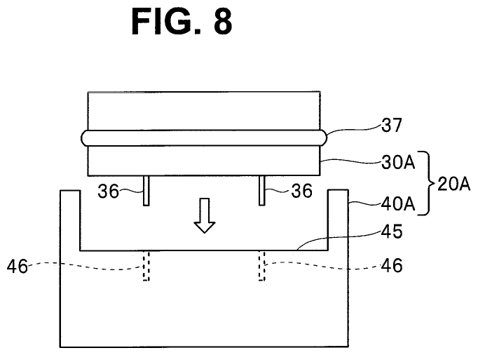

FIG. 8 is a view showing an example of a configuration of an image pickup module according to a second embodiment.

An image pickup module 20A is configured by a first case 30A and a second case 40A.

The first case 30A has two connection pins 36 (first electric contacts), and a seal portion (first seal) 37. Note that inside of the first case 30A, a substrate 31, an image pickup device 32, LEDs 33, and a transmitter 34 are provided, as in FIG. 2.

The second case 40A has a recessed portion 45 into which the first case 30A is fitted, and two connection connectors 46 (second electric contacts or a plurality of receiver channels) to which the two connection pins 36 are respectively connected.

The second case 40 includes the plurality of receiver channels 46. The first one of the second connectors 43 is located in a first channel of the plurality of receiver channels 46, and the second one of the second connectors 43 is located in a second channel of the plurality of receiver channels 46.

The first one of the first connectors 36 is a first connector pin inserted into the first channel and in contact with the first one of the second connectors 43, and the second one of the first connectors 36 is a second connector pin inserted into the second channel and in contact with the second one of the second connectors 43.

The first seal 37 can be located between a first periphery surface of the first case 30 and a seating surface of the second case 40 to seal a portion of a joint between the first case 30 and the second case 40.

The connection pins 36 are provided on an undersurface of the first case 30A, and are connected to the connection connectors 46 when the first case 30A is fitted into the recessed portion 45 of the second case 40A.

Note that in the present embodiment, the first case 30A has the connection pins 36, whereas the second case 40A has the connection connectors 46, but without being limited to this, the first case 30A may have the connection connectors, and the second case 40A may have the connection pins.

The seal portion 37 is provided to surround an outer peripheral portion of the first case 30A. The outer peripheral portion of the first case 30A configures a joining portion that is joined to the second case 40A. An outer peripheral surface of the joining portion is covered with an insulator. The seal portion 37 ensures watertightness of the first case 30A and the second case 40A when the first case 30A is fitted into the recessed portion 45 of the second case 40A. At least one of a portion of an outer peripheral surface of the power supply connection and a portion of an outer peripheral surface of the ground connection can be covered with an insulator. The first case 30 can include a first substrate 31, and the second case can include the second substrate 41. The first substrate 31 is electrically connected to the second substrate 41 via the power supply connection.

In the image pickup module 20 of the first embodiment, the seal portion 44 is provided on each of the plurality of connection pins 43. In contrast to this, the image pickup module 20A of the present embodiment can be provided with the one sealing portion 37 even if the image pickup module 20A has the plurality of connection pins 36, so that cost can be reduced as compared with the first embodiment.

The present disclosure is not limited to the aforementioned embodiments, and various changes, alterations and the like can be made in the range without changing the gist of the present disclosure.
Example 1. An image pickup module for endoscope comprising:
   a first case inside of which an image pickup device is disposed;
   a second case inside of which a battery configured to supply power to the image pickup device is disposed;
   a joining portion that is provided in one case of the first and the second cases, and configured to be inserted into another case of the first and the second cases and to removably connect the image pickup device and the battery; and
   a seal portion provided at the joining portion and configured to seal the other case in a watertight manner.
Example 2. The image pickup module for endoscope according to Example 1,
   wherein the seal portion further seals the one case in a watertight manner.
Example 3. The image pickup module for endoscope according to Example 1,
   wherein the one case is the second case.
Example 4. The image pickup module for endoscope according to Example 3,
   wherein the joining portion is a power supply pin connected to the battery.
Example 5. The image pickup module for endoscope according to Example 1,
   wherein an LED is provided in the first case, and the LED is supplied with power by the battery.
Example 6. The image pickup module for endoscope according to Example 1,
   wherein a transmitter is provided in the first case, and the transmitter is supplied with power by the battery.
Example 7. The image pickup module for endoscope according to Example 6,
   wherein the transmitter transmits an electrical signal outputted from the image pickup device to an external receiver.
Example 8. The image pickup module for endoscope according to Example 4,
   wherein the seal portion is a packing provided at the power supply pin.
Example 9. The image pickup module for endoscope according to Example 8,
   wherein the packing is an O-shaped ring.
Example 10. The image pickup module for endoscope according to Example 1,
   wherein an outer peripheral surface of the joining portion is covered with an insulator.
Example 11. The image pickup module for endoscope according to Example 10,
   wherein the joining portion includes a first electric contact,
   the other case includes a second electric contact that electrically continues to the first electric contact, and
   the image pickup device and the battery are connected to each other via the first electric contact and the second electric contact.
Example 12. The image pickup module for endoscope according to Example 11,
   wherein the other case is the first case.
Example 13. The image pickup module for endoscope according to Example 11,
   wherein the other case is the second case.
Example 14. The image pickup module for endoscope according to Example 10,
   wherein a transmitter is provided in the first case, and the transmitter is supplied with power by the battery.
Example 15. The image pickup module for endoscope according to Example 14,
   wherein the transmitter transmits an electrical signal outputted from the image pickup device to an external receiver.
Example 16. An insertion portion for endoscope including
   a tube in which an image pickup module is fixed to a distal end,
   the image pickup module comprising
   a first case inside of which an image pickup device is disposed,
   a second case inside of which a battery configured to supply power to the image pickup device is disposed,
   a joining portion that is provided in one case of the first and the second cases, and configured to be inserted into another case of the first and the second cases and to removably connect the image pickup device and the battery, and
   a seal portion provided at the joining portion and configured to seal the other case in a watertight manner.
Example 17. An endoscope including
   an insertion portion including a tube in which an image pickup module is fixed to a distal end,
   the image pickup module comprising
   a first case inside of which an image pickup device is disposed,
   a second case inside of which a battery configured to supply power to the image pickup device is disposed,
   a joining portion that is provided in one case of the first and the second cases, and configured to be inserted into another case of the first and the second cases and to removably connect the image pickup device and the battery, and
   a seal portion provided at the joining portion and configured to seal the other case in a watertight manner.
Example 18. The endoscope according to Example 17,
   wherein an LED is provided in the first case, and the LED is supplied with power by the battery.
Example 19. The endoscope according to Example 17,
   wherein a transmitter is provided in the first case, and the transmitter is supplied with power by the battery.
Example 20. The endoscope according to Example 17,
   wherein the transmitter transmits an electrical signal outputted from the image pickup device to an external receiver.

What is claimed is:

1. An image pickup module, the image pickup module comprising:
a first case including an image sensor and one or more of first connectors;
a second case including a power source and one or more of second connectors; and
wherein a first one of the first connectors is electrically connected to a first one of the second connectors to form a power supply connection between the image sensor and the power source.

2. The image pickup module according to claim 1, wherein a second one of the first connectors is electrically connected to a second one of the second connectors to form a ground connection between the image sensor and the power source.

3. The image pickup module according to claim 1, further comprising a first seal connected to the first one of the second connectors,
wherein the first seal contacts a surface of the first case to prevent a liquid from entering an interior of the first case.

4. The image pickup module according to claim 3, wherein the first seal contacts a surface of the second case to prevent a liquid from entering an interior of the second case.

5. The image pickup module according to claim 3, wherein the first seal is an O-shaped ring.

6. The image pickup module according to claim 2, wherein an exterior surface of the first case includes a plurality of receiver channels,
wherein the first one of the first connectors is located in a first channel of the plurality of receiver channels and the second one of the first connectors is located in a second channel of the plurality of receiver channels,
wherein the first one of the second connectors is a first connector pin inserted into the first channel and in contact with the first one of the first connectors and the second one of the second connectors is a second connector pin inserted into the second channel and in contact with the second one of the first connectors,
wherein the image pickup module further comprises a first seal connected to the first connector pin and a second seal connected to the second connector pin, and
wherein the first seal contacts a first surface portion of the first case to seal the first channel and the second seal contacts a second surface portion of the first case to seal the second channel.

7. The image pickup module according to claim 2, wherein the second case includes a plurality of receiver channels,
wherein the first one of the second connectors is located in a first channel of the plurality of receiver channels and the second one of the second connectors is located in a second channel of the plurality of receiver channels,
wherein the first one of the first connectors is a first connector pin inserted into the first channel and in contact with the first one of the second connectors and the second one of the first connectors is a second connector pin inserted into the second channel and in contact with the second one of the second connectors, and
wherein the image pickup module further comprises a first seal located between a first periphery surface of the first case and a seating surface of the second case to seal a portion of a joint between the first case and the second case.

8. The image pickup module according to claim 1, wherein the first case further includes an LED, and wherein the LED is supplied with power from the power source via the power supply connection.

9. The image pickup module according to claim 1, wherein the first case further includes a transmitter, and wherein the transmitter is supplied with power from the power source via the power supply connection.

10. The image pickup module according to claim 9, wherein the transmitter is configured to transmit an electrical signal outputted from the image sensor.

11. The image pickup module according to claim 1, wherein at least one of a portion of an outer peripheral surface of the power supply connection and a portion of an outer peripheral surface of the ground connection is covered with an insulator.

12. The image pickup module according to claim 1, wherein the first case includes a first substrate and the second case includes a second substrate, and
wherein the first substrate is electrically connected to the second substrate via the power supply connection.

13. An endoscope, comprising,
an insertion portion including a distal end; and
the image pickup module according to claim 1,
wherein the image pickup module is reversibly attached to the distal end of the insertion portion.

14. The endoscope according to claim 13, wherein the first case further includes an LED and wherein the LED is supplied with power from the power source via the power supply connection.

15. The endoscope according to claim 13, wherein the first case further includes a transmitter, and wherein the transmitter is supplied with power from the power source via the power supply connection.

16. The endoscope according to claim 13, wherein the transmitter is configured to transmit an electrical signal outputted from the image sensor.

17. The endoscope according to claim 13, wherein the first case and the second case extend along a longitudinal direction of the insertion portion, and wherein the first case and the second case are stacked in a stacking direction that intersects the longitudinal direction.

18. The endoscope according to claim 13, wherein a longest dimension of the image sensor and a longest dimension of the power source both extend along the longitudinal direction.

19. The image pickup module according to claim 1, further comprising a first seal connected to the first one of the first connectors,
wherein the first seal contacts a surface of the second case to prevent a liquid from entering an interior of the second case.
